(19) United States
(12) Patent Application Publication
(10) Pub. No.: US 2006/0178574 A1
(43) Pub. Date: Aug. 10, 2006

(54) ECG electrode arrangement for MR applications

(76) Inventors: Daniel Fischer, Erlangen (DE); Werner Lindstedt, Kalchreuth (DE); Stefan Merkel, Erlangen (DE)

Correspondence Address: SIEMENS CORPORATION;INTELLECTUAL PROPERTY DEPARTMENT, 170 WOOD AVENUE, SOUTH, ISELIN, NJ, 08830, US

(21) Appl. No.: 11/345,723

(22) Filed: Feb. 2, 2006

(30) Foreign Application Priority Data
Feb. 2, 2005 (DE) 10 2005 004 859.5

(51) Int. Cl. A61B 5/04

(52) U.S. Cl. 600/372; 600/509

(57) ABSTRACT

ECG electrode arrangement for MR applications in alternating magnetic fields, in particular triggering with MR applications, with a correction loop being provided, which is practically congruent with each measuring loop made of electrode supply lines and insulated therefrom, the induction voltage with reverse polarity of which overlays the output voltage of the measuring loop.

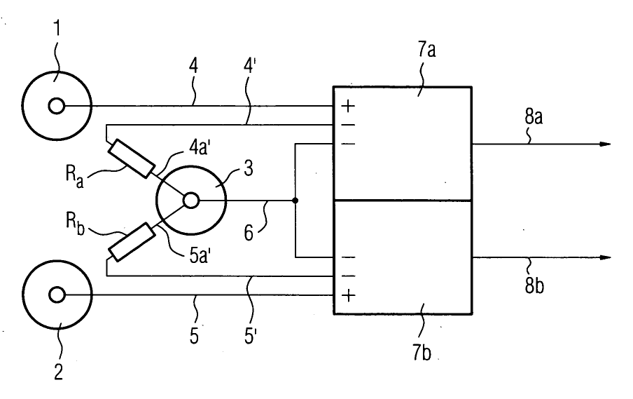

### CROSS REFERENCE TO RELATED APPLICATIONS

This application claims priority to the German application No. 10 2005 004 859.5, filed Feb. 2, 2005 which is incorporated by reference herein in its entirety.

### FIELD OF INVENTION

The invention relates to an ECG electrode arrangement for applications in alternating magnetic fields, in particular for triggering with MR applications

### BACKGROUND OF INVENTION

Faulty triggering occurs in many patients during ECG triggering with MR. Highly accurate mathematical methods exist for the precise detection of R peaks, which are essential identifiers of every ECG. In the case of MR, detection is however made more difficult in that the electrodes or lines intercept interferences which are generated by gradient and/or high-frequency pulses. Coupling thus naturally depends on the spatial arrangement of the electrodes.

### SUMMARY OF INVENTION

In the past this problem was not solved completely satisfactorily. The use of mathematical methods does not always enable triggering to take place at the exact time of the R peak, whereby a certain delay can still be accepted if necessary. It has already been proposed however that the influence of gradients should be re-determined for each patient and then be subtracted at the time of measurement. This however requires communication for the purpose of ECG data acquisition, said communication not having previously been available.

The article by M. Laudon et al.: “Minimizing Inference from Magnetic Resonance Imagers During Electrocardiography”, IEEE Transactions on Biomedical Engineering, Vol. 45, No.2, February 1998, pages 160 to 164, addresses the problem of interference during ECG recording, when the patient is located in a magnetic resonance unit. In a test setup, electrodes are attached to a test object, said electrodes being routed to a first amplifier via a low-pass filter, an external conductor loop is similarly connected to a low -pass filter, the signal is then routed to a second amplifier, the signals of the two amplifiers are fed to a differential amplifier. The output signal resulting from this test arrangement contains a good ECG signal with minimal magnetic interference.

DE 33 46 866 A1 discloses a device for the application of ECG electrodes. This device includes a number of ECG electrodes with an in-line wiring harness. It is proposed that ECG electrodes should be arranged at irregular intervals to ensure that the R peak of the electrocardiogram is particularly efficiently recorded.

An object underlying the invention is to create an ECG electrode arrangement which can also be used in magnetic alternating fields, in particular magnetic resonance tomographs, without unwanted induction voltages overlaying the measurement voltages.

To solve this problem, provision is made according to the invention for a correction loop to be arranged practically congruent with each measuring loop made of electrode supply leads and insulated therefrom, the induction voltage with reverse polarity of said correction loop overlaying the output voltage of the measuring current loop.

In the simplest case, the development according to the invention is thereby designed such that a correction line is arranged parallel to each voltage-carrying electrode line and insulated therefrom, said correction line being linked to the return line out from a reference electrode by a spur line branching off at the electrode end.

If correction resistors corresponding to the skin resistance between the electrode and the reference electrode are also arranged in the spur lines, as is provided for according to an additional feature of the present invention, actual correction measuring loops result, in which the same interference voltages are induced practically identically by the gradient and/or high-frequency pulses, so that the interferences can be almost completely eliminated by corresponding overlaying with reverse signs.

This elimination of the induced interference voltage allows the already addressed mathematical methods for timely detection of the R peak to be applied to ECG signals, so that they are no longer adversely affected as before by the interference signals in the alternating magnetic fields.

### BRIEF DESCRIPTION OF THE DRAWING

Further advantages, features and details of the invention are set down in the subsequent description of an exemplary embodiment as well as with reference to the drawings, which represent a schematic representation of an ECG electrode arrangement according to the invention, with reference to which the principle of the invention is to be explained. A precise wiring arrangement is not shown here.

### DETAILED DESCRIPTION OF INVENTION

The ECG electrode arrangement displayed includes two electrodes 1 and 2 which can be attached to the skin of the patient as well as a reference electrode 3. The measuring lines 4 and 5 to the electrodes 1 and 2 are connected respectively to the positive input of the subtracting measuring amplifier 7a or 7b. In accordance with the invention, correction lines 4′ and 5′ are arranged parallel and directly adjacent to the measuring lines 4 and 5, said correction lines 4′, 5′ reaching the region of the electrodes 1 and 2 and being connected from there to the return line 6 via spur lines 4a′ and 5a′, in which resistors R and R corresponding to the skin resistance between the electrodes 1, 2 and the reference electrode 3 are arranged. This means that the correction lines with the spur lines and the return line form a measuring loop of exactly the same size and at an identical location to that of the electrodes with their supply leads, so that the corresponding overlaying of the voltages of the measuring loops and the correction measuring loops in the subtractor and amplifier. 7a, 7b allows the interference voltages induced in these loops by magnetic alternating fields to be completely eliminated. Lines 8a, 8b for evaluating the first or the second outgoing line are indicated at the output of the subtractor and amplifier 7a, 7b.

The invention is not limited to the exemplary embodiment shown, in that it does not depend specifically on the use of only three electrodes. The electrode lines and the correction lines can be designed in any manner, provided it is ensured that on the one hand they can be as congruent as possible with the body of the patient and on the other hand they are of course insulated from one another.

1-3. (canceled)

4. An ECG electrode arrangement for applications in alternating magnetic fields, comprising:
at least one measuring loop comprising electrode lines; and
a correction loop assigned to the measuring loop, the correction loop arranged essentially congruent with and insulated from the measuring loop, wherein an induction voltage of the correction loop has a reverse polarity relative to an output voltage of the measuring loop, and the output voltage is superimposed with the induction voltage.

5. An ECG electrode arrangement, comprising:
at least one measuring electrode line including at least one measuring electrode;
a reference electrode; and
a correction measuring line arranged parallel to and insulated from the measuring electrode line, the correction measuring line connected to a return line of the reference electrode by a spur line branching off at an electrode end of the reference electrode.

6. The ECG electrode arrangement according to claim 5, further comprising a correction resistor arranged in the spur line and having a resistance according to a skin resistance present between the measuring electrode and the reference electrode when the ECG electrode arrangement is connected to the skin of a person under examination.
